(19) United States
(12) Patent Application Publication
(10) Pub. No.: US 2019/0021689 A1
(43) Pub. Date: Jan. 24, 2019

(54) X-RAY IMAGING APPARATUS

(71) Applicant: Shimadzu Corporation, Kyoto (JP)

(72) Inventors: Dai HIROSE, Kyoto (JP); Tetsu NAKAYAMA, Kyoto (JP); Koki YOSHIDA, Kyoto (JP)

(21) Appl. No.: 16/033,715

(22) Filed: Jul. 12, 2018

(30) Foreign Application Priority Data
Jul. 19, 2017 (JP) 2017-140059

(51) Int. Cl. A61B 6/00; A61B 6/08; G01T 1/02

(52) U.S. Cl. CPC A61B 6/584; G01T 1/02; A61B 6/08

(57) ABSTRACT

An X-ray imaging apparatus is provided with a distance sensor for measuring an SSD which is a distance between a focal point of the X-ray tube and a surface of a subject M, an input unit for setting a reference SSD associated with an incident dose in which imaging is allowed, and a comparator for comparing an SSD at the time of imaging associated with the incident dose obtained by using the SD measured by the distance sensor and a reference SSD set by the input unit.

### TECHNICAL FIELD

The present invention relates to an X-ray imaging apparatus for performing X-ray imaging, and more particularly to a technique for reducing X-ray exposure to a subject.

### BACKGROUND ART

As an apparatus of this type, an example will be described by exemplifying a conventional angiography device for acquiring an angiogram by administering a contrast agent to a subject. The angiography device is provided with an X-ray tube for irradiating X-rays and an X-ray detector for detecting the X-rays. With both of them facing each other, by performing the angle adjustment of the rotation (hereinafter also referred to as “oblique”) of a subject about the body axis of the subject which is a patient and the rotation (hereinafter also referred to as “sagittal”) of the subject about the body axis direction thereof, or performing a height adjustment of the examination table (top board of the examination table) having the top board on which the subject is placed, the state of the blood vessels of the subject placed on the top board can be observed.

It should be noted that in this specification, the term “imaging” includes a case where an X-ray image is acquired by irradiating X-rays with a strong dose and a case where an moving image is displayed by sequentially displaying X-ray images by continuously irradiating X-rays with a dose lower than the former case (fluoroscopy).

In an examination and a medical treatment using an angiography device, it is very important to reduce the X-ray exposure amount to a subject. Conventionally, it has been devised to reduce the exposure as follows. For example,

(1) The exposure is reduced by changing the X-ray conditions (imaging conditions), such as, e.g., a tube voltage of an X-ray tube and a pulse width (exposure time) of X-ray irradiation.

(2) In order to prevent image quality degradation due to the above approach (1), digital image processing such as addition processing is performed to reduce the exposure while maintaining the image quality.

(3) By providing information to a user (operator) such as providing a dose map of a patient, the subsequent imaging is performed while shifting so that X-rays are irradiated to an area which has been less exposed.

In the present invention, as another approach different from the above approaches (1) to (3), the exposure is reduced by moving a holding mechanism of the X-ray tube and the X-ray detector. Note that the distance between the focal point of the X-ray tube and the subject is called SOD (Source Object Distance), in particular the distance between the focal point of the X-ray tube and the surface of the subject is also called SSD (Source Surface Distance). These distances are measured using a distance sensor composed of an optical sensor (see, for example, Patent Document 1).

### PRIOR ART DOCUMENT

### Patent Document

### [Patent Document 1]

### Japanese Unexamined Patent Application Publication No. 2016-119939

### SUMMARY OF THE INVENTION

### Problems to be Solved by the Invention

However, in order to suppress exposure to a subject, it is necessary to suppress the incident dose irradiated to the surface (skin) of the subject. For that purpose, it is important to keep the aforementioned SSD large.

That is, as the SSD becomes smaller, the incident dose irradiated to the surface (skin) of the patient on the tube side of the X-ray tube increases, which increases the exposure. For this reason, from the viewpoint of reducing the exposure, it is preferable to keep the SSD as large as possible.

Here, the “incident dose” is a dose at the skin position of the patient, and the incident dose decreases as the SSD increases in inverse proportion to the square of the SSD. The unit of the incident dose is [Gy]. Further, an “area dose” is a dose calculated by multiplying the aforementioned incident dose by the irradiation area, and the area dose is constant regardless of the distance like the SSD. The unit of the area dose is [Gy·m²]. Actually, since the irradiation area changes as the SSD changes, the area dose can be regarded as a total dose, and therefore the area dose can be obtained by measuring the area dose with a dosimeter or from the X-ray conditions (the tube current of the X-ray tube, the pulse width of the X-ray irradiation, etc.).

However, the subject M is not a spherical shape but a plate-like shape as shown in the rotation (sagittal rotation) in the body axis direction shown in FIG. 12A and the rotation (oblique rotation) about the body axis shown in FIG. 12B. Therefore, when both the sagittal and the oblique reach a deep depth with reference to the irradiation immediately above or immediately below, the SSD becomes smaller. For this reason, a user (operator) has to operate with caution so that the SSD does not become too small.

The present invention has been made in view of such circumstances, and it is an object of the present invention to provide an X-ray imaging apparatus capable of suppressing an incident dose.

### Means for Solving the Problems

In order to attain such an object, the present invention has the following configuration.

That is, an X-ray imaging apparatus according to the present invention is an X-ray imaging apparatus for performing X-ray imaging. The X-ray imaging apparatus is provided with an X-ray tube configured to irradiate X-rays, SSD deriving means configured to derive an SSD which is a distance between a focal point of the X-ray tube and a surface of a subject, setting means configured to set a reference physical quantity associated with an incident dose in which imaging is allowed, comparing means configured to compare a physical quantity at the time of imaging associated with an incident dose derived using the SSD by the SSD deriving means and the reference physical quantity set by the setting means, and control means configured to perform a predetermined operation using a comparison result by the comparing means.

[Functions and Effects] According to the X-ray imaging apparatus of the present invention, the X-ray imaging apparatus is provided with SSD deriving means configured to obtain the SSD which is the distance between the focal point of the X-ray tube and the surface of the subject. The X-ray imaging apparatus is further provided with setting means configured to set a reference physical quantity associated with the incident dose in which imaging is allowed and comparing means configured to compare the physical quantity at the time of imaging associated and the incident dose obtained by using the SSD derived by the SSD deriving means with the reference physical quantity set by the setting means. By providing such setting means and comparing means, it can be determined by the comparing means whether or not the physical quantity at the time of imaging associated with the incident dose obtained by using the SSD falls within the imaging allowable range with reference to the physical quantity. By providing the control means configured to perform a predetermined operation using the comparison result by the comparing means, it is possible to suppress the incident dose by carrying out the predetermined operation.

As the predetermined operation, the following operations can be exemplified. The control means performs a moving operation (one example of the former predetermined operation) of the X-ray tube so that the physical quantity at the time of imaging falls within an imaging allowable range with reference to the reference physical quantity by using the comparison result by the comparing means. Alternatively, the control means notifies (one example of the latter predetermined operation) that the physical quantity at the time of imaging deviates the imaging allowable range with reference to the reference physical quantity when the physical quantity deviates the imaging allowable range by using the comparison result by the comparing means. Note that that the former example and the latter example (notification) may be combined. A specific example of the former example of the predetermined operation will be described later.

An example of the physical quantity (associated with the incident dose), which is a target of the comparison, is the aforementioned SSD. When the physical quantity is an SSD, the comparing means compares the SSD at the time of imaging and a reference SSD in which imaging is allowed, and the control means performs a moving operation of the X-ray tube such that the SSD at the time of imaging does not fall below the reference SSD by using the comparison result by the comparing means. When the SSD at the time of imaging falls below the reference SSD, the SSD becomes too small and therefore the incident dose deviates from the imaging allowable range and increases. Therefore, by performing the moving operation of the X-ray tube such that the SSD at the time of imaging does not fall below the reference SSD, the SSD can be kept large so that the incident falls within the imaging allowable range, which can suppress the incident dose. Note that the physical quantity (associated with the incident dose), which is a target of the comparison, may be an incident dose. In the case of the incident dose, it can be obtained by multiplying a value obtained by dividing the product of the area dose [Gy·m²] measured by a dosimeter or the tube current value [A] and the imaging time [s] by the square [m²] of the SSD by a factor.

The aforementioned SSD deriving means is a distance sensor for measuring an SSD. The SSD deriving means is not limited to a distance sensor and the SSD may be calculated by calculation. For example, the SSD deriving means may calculate the SSD from the relative position between the shape data of the subject captured in advance and the focal point of the X-ray tube. Further, the SSD deriving means may calculate the SSD from a relative position between a model (a model which is imitated by an oval spherical shape imitating a head of a subject or a plate-like shape imitating a middle of a subject) imitating the subject and the focal point of the X-ray tube.

A specific embodiment of the former example of the predetermined operation will be described. In cases where the physical quantity (associated with the incident dose) is the SSD, the comparing means compares the SSD at the time of imaging and the reference SSD in which imaging is allowed, and the control means makes a notification that the SSD at the time of imaging falls below the reference SSD when the SSD falls below the reference SSD and simultaneously performs a moving operation of the X-ray tube so that the SSD at the time of imaging does not fall below the reference SSD by using the comparison result by the comparing means. It is possible to automatically notify a user that the SSD at the time of imaging falls below the reference SSD and perform the moving operation of the X-ray tube so that the SSD at the time of imaging does not fall below the reference SSD.

As the moving operation of the X-ray tube, a rotation operation of the X-ray tube is primarily exemplified. That is, the control means makes the aforementioned notification and simultaneously stops the rotation operation of the X-ray tube. With this, the X-ray tube is prevented from moving to a deep angle with reference to the irradiation immediately above or immediately below. As described in the section “Problem to be Solved by the Invention”, since the subject does not have a spherical but has a plate-like shape, when both the sagittal and the oblique reaches a deep depth with reference to the irradiation immediately above or immediately below, the SSD becomes small. However, by stopping the rotation operation of the X-ray tube simultaneously with the aforementioned notification, the SSD at the time of imaging keeps the reference SSD so as not to rotate to a deep angle.

As the moving operation of the X-ray tube, it is not limited to the rotation operation of the X-ray tube. For example, the control means may make the notification and simultaneously retract the X-ray tube so as to keep the X-ray tube away from the subject. By retracting the X-ray tube, the SSD at the time of imaging increases to give a margin with respect to the reference SSD. Therefore, it becomes possible to continuously perform the operation of the X-ray tube (for example, rotation operation of the X-ray tube in the same direction as the direction immediately before the notification) while keeping the lower limit of the reference SSD, and therefore the operation of the X-ray tube can be continued.

Note that the control target is not limited to the X-ray tube, and the operation of the top board for placing a subject thereon may be controlled. That is, the control means makes the notification and simultaneously translates (when the placement surface is horizontal and the X-ray tube is positioned below the top board, the top board is moved upward, and when the placement surface is horizontal and the X-ray tube is positioned above the top board, the top board is moved downward) the top board in a direction perpendicular to the placement surface of the top board so that the subject is moved away from the X-ray tube to continuously perform the rotation operation of the X-ray tube in the same direction as a direction immediately before the notification. In the same manner as in the case of retracting the X-ray tube away from the subject, by translating the top board in a direction perpendicular to the placement surface of the top board, the SSD at the time of imaging increases to give a margin with respect to the reference SSD. Therefore, it becomes possible to continuously perform the rotation operation of the X-ray tube while keeping the lower limit of the reference SSD.

Further, as to the setting of the reference SSD by the aforementioned setting means, it may be performed in a plurality of stages. More specifically, when “n” is an integer of 2 or more, and “k” is an integer satisfying 2≤k≤n, the above setting means sets the values of the reference SSD in descending order in a plurality of stages A₁, A₂, . . . , Aₖ, . . . , A₍ₙ₋₁₎, Aₙ (that is, A₁>A₂> . . . >Aₖ> . . . >A₍ₙ₋₁₎>Aₙ). The control means performs a process of notifying that the SSD at the time of imaging falls below the reference SSD(A₁) when the SSD at the time of imaging falls below the reference the SSD(A₁) and simultaneously continuously performing the rotation operation of the X-ray tube until the SSD reaches the reference SSD(A₂) reset by the setting means, and thereafter repeatedly performs a process of notifying that the SSD at the time of imaging falls below the reference SSD(A₍ₖ₋₁₎) when the SSD at the same time of imaging falls below the reference SSD(A₍ₖ₋₁₎) and simultaneously continuously performing the rotation operation of the X-ray tube until the SSD reaches the reference SSD(Aₖ) reset by the setting means. Further, the control means notifies that the SSD at the time of imaging falls below the reference SSD(Aₙ) when the SSD falls below the reference SSD(Aₙ) and simultaneously stops the rotation operation of the X-ray tube. In this way, when it is desired to rotate the X-ray tube to a deeper angle with reference to the irradiation from immediately above or immediately below, the reference SSD is set in descending order in a plurality of stages.

In particular, when n=2, the value of the reference SSD is set to A₁ and A₂ in descending order in two stages (that is, A₁>A₂). The control means makes a notification that the SSD at the time of imaging falls below the reference SSD(A₁) when the SSD at the time of imaging falls below the SSD(A₁) and simultaneously continuously performs the rotation operation of the X-ray tube until the SSD at the time of imaging reaches the reference SSD(A₂) reset by the setting means, and makes a notification that the SSD at the time of imaging falls below the reference SSD(A₂) when the SSD at the time of imaging falls below the SSD(A₂) and simultaneously stops the X-ray tube. In this way, when limiting to n=2, in cases where it is desired to rotate the X-ray tube to a deeper angle with reference to the irradiation from immediately above or immediately below, the reference SSD is in descending order in two steps.

In the latter example (notification) of the predetermined operation, in cases where the imaging apparatus is provided with selection means configured to select any one of operation mode from a plurality of moving operation modes of the X-ray tube, the X-ray detector for detecting X-rays and the top board for placing the subject thereof, the following embodiments can be exemplified.

As the former embodiment, the control means performs the aforementioned notification and simultaneously perform the moving operation of the X-ray tube, the X-ray detector, or the top board in accordance with the moving operation mode selected by the aforementioned selection means. In the case of the former embodiment, by performing the selection in advance before the operation of the apparatus, effects can be exerted in which the control means can perform the notification and simultaneously quickly perform the moving operation of the X-ray tube, the X-ray detector, or the top board in accordance with the moving operation mode selected by the aforementioned selection means.

As the latter embodiment, the control means performs the selection by the aforementioned selection means after the notification, and thereafter performs the moving operation of the X-ray tube, the X-ray detector, or the top board in accordance with the moving operation mode selected by the aforementioned selection means. In the latter embodiment, it is advantageous when a user wishes to select each moving operation mode from time to time after the user receives the notification.

### Effects of the Invention

According to the X-ray imaging apparatus of the present invention, it is provided with SSD deriving means configured to derive an SSD which is a distance between a focal point of the X-ray tube and a surface of a subject, setting means configured to set a reference physical quantity associated with an incident dose which is allowed for imaging, and comparing means configured to compare a physical quantity at the time of imaging associated and an incident dose derived by using the SSD obtained by the SSD deriving means with the reference physical quantity set by the setting means. By providing these means, it can be determined by the comparing means whether or not the physical quantity at the time of imaging associated with the incident dose obtained by using the SSD falls within the imaging allowable range with reference to the physical quantity. By providing the control means configured to perform a predetermined operation using the comparison result by the comparing means, it is possible to suppress the incident dose by carrying out the predetermined operation.

### BRIEF DESCRIPTION OF THE DRAWINGS

FIG. 1 is a side view of an X-ray imaging apparatus equipped with a multi-joint arm according to each Example.

FIG. 2 is a front view of the X-ray imaging apparatus shown in FIG. 1.

FIG. 3 is a block diagram of an X-ray imaging apparatus according to Examples 1 to 6.

FIG. 4 is a schematic diagram showing a combination of a rotation operation amount of oblique, a rotation operation amount of sagittal, and an operation amount of a movable top board in a vertical direction, which do not fall below the reference SSD in which imaging is allowed.

FIG. 5 is a display mode of a monitor showing a range of a rotation operation amount of oblique and a range of a rotation operation amount of sagittal at a certain height of a movable top board, which do not fall below the reference SSD in which imaging is allowed.

FIG. 6A and FIG. 6B are front views of an X-ray tube and an X-ray detector showing a change of an SSD by an oblique rotation, which is a moving operation of an X-ray tube according to Example 1.

FIG. 7A and FIG. 7B are front views of an X-ray tube and an X-ray detector showing a change of an SSD by an oblique rotation, which is a moving operation of an X-ray tube according to Example 2.

FIG. 8A and FIG. 8B are front views of an X-ray tube and an X-ray detector showing a change of an SSD by an oblique rotation, which is a moving operation of a movable top board according to Example 3.

FIG. 9A and FIG. 9B are front views of an X-ray tube and an X-ray detector showing a change of an SSD by an oblique rotation, which is a moving operation of an X-ray tube according to Example 4.

FIG. 10 is a display mode of a monitor selection screen according to Examples 5 and 6.

FIG. 11 is a block diagram of an X-ray imaging apparatus according to Example 7 in the case where an incident dose is obtained from an area dose measured with a dosimeter.

FIG. 12A is a schematic view for explaining rotation (sagittal rotation) in a body axis direction, and FIG. 12B is a schematic diagram for explaining rotation (oblique rotation) about a body axis.

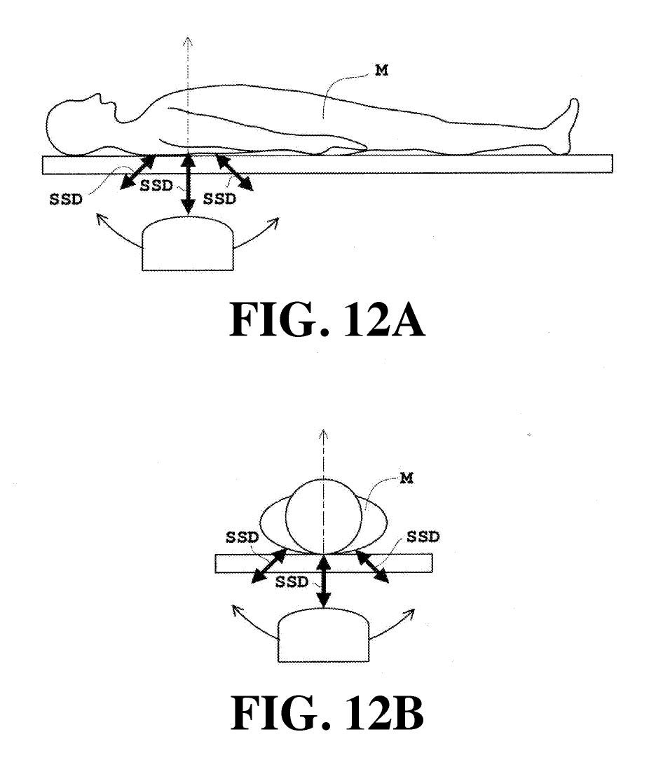

### DESCRIPTION OF PREFERRED EMBODIMENTS

### Example 1

Hereinafter, Example 1 of the present invention will be described with reference to the drawings.

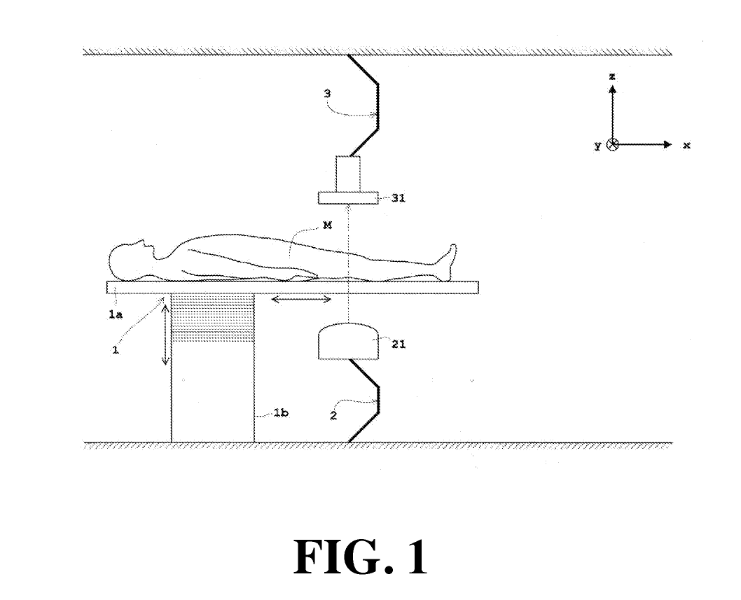

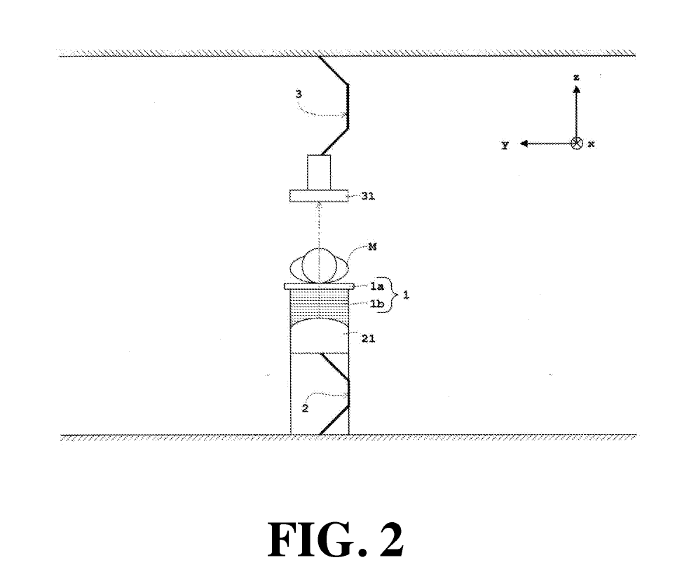

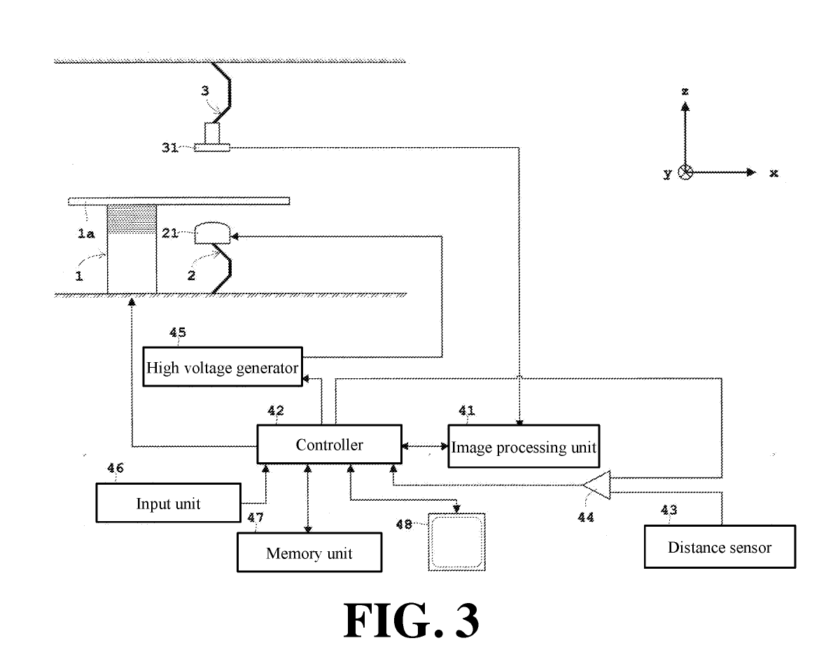

FIG. 1 is a side view of an X-ray imaging apparatus equipped with a multi-joint arm according to each Example. FIG. 2 is a front view of the X-ray imaging apparatus shown in FIG. 1. FIG. 3 is a block diagram of an X-ray imaging apparatus according to Examples 1 to 6.

As shown in FIG. 1 and FIG. 2, the X-ray imaging apparatus according to Example 1 is provided with an examination table 1 having a movable top board 1a for placing a subject M thereon which is movable in a longitudinal direction with respect to a main body 1b, a multi-joint arm 2 for an X-ray tube, and a multi-joint arm 3 for an X-ray detector, and as shown in FIG. 3, the X-ray imaging apparatus is further provided with an image processing unit 41, a controller 42, a distance sensor 43, a comparator 44, a high voltage generator 45, an input unit 46, a memory unit 47, and a monitor 48, which can also be applied to Examples 2 to 7 to be described later.

The multi-joint arm 2 for the X-ray tube is supported on the floor surface (xy plane in the drawing), and the tip end arm supports the X-ray tube 21. In FIG. 1 and FIG. 2, the multi-joint arm 2 is composed of three arms in which ends thereof are connected. By rotating the end portion of each arm as a fulcrum, it can be moved in the horizontal direction (xy-direction in the figure), moved upward or downward (in the z-direction in the figure), rotated (sagittal rotation) in the subject axis (X axis in the figure) of the subject, or rotated (oblique rotation) about the body axis of the subject M. As the arm constituting the multi-joint arm 2 moves in each direction including the rotatory movement, the X-ray tube 21 supported by the multi-joint arm 2 also moves in the same direction as the multi-joint arm 2. The number of arms constituting the multi-joint arm 2 is not limited to three as shown in FIG. 1 and FIG. 2, and may be three or more, or may be two.

The multi-joint arm 3 for the X-ray detector is hung and supported from the ceiling surface (xy plane in the figure), and the tip arm supports the X-ray detector 31. In the same manner as in the multi-joint arm 2 for the X-ray tube, the multi-joint arm 3 is composed of three arms in which the ends of arms are connected. By rotating the end portion of each arm as a fulcrum, it can be moved in the horizontal direction (xy-direction in the figure), moved upward and downward in the vertical direction (in the z-direction in the figure), rotated (sagittal rotation) in the subject axis (X-axis in the figure) of the subject, or rotated (oblique rotation) about the body axis of the subject M. As the arm constituting the multi-joint arm 3 moves in each direction including the rotatory movement, the X-ray tube 31 supported by the multi-joint arm 3 also moves in the same direction as the multi-joint arm 3. In the same manner as in the multi-joint arm 2 for the X-ray tube, the number of arms constituting the multi-joint arm 3 is not limited to three as shown in FIG. 1 and FIG. 2, and may be three or more, or may be two.

As described above, the X-ray tube 21 and the X-ray detector 31 are supported by independent multi-joint arms 2 and 3, respectively, and the X-ray tube 21 and the X-ray detector 31 are independently driven. Then, the controller 42 (see FIG. 3) controls so that the movements of the X-ray tube 21 and the X-ray detector 31 are synchronized with each other, and controls so that the X-ray tube 21 and the X-ray detector 31 face via the movable top board 1a. The X-ray detector 31 is not particularly limited as long as it is a normally used X-ray detector as exemplified by a flat panel type X-ray detector (FPD: Flat Panel Detector) or an image intensifier (I.I).

The body 1b of the examination table 1 can be raised and lowered in the vertical direction, so that the movable top board 1a is configured so as to be moved upward and downward in the vertical direction. As shown in FIGS. 1 and 2, in the case of imaging the subject M in a recumbent posture, the placement surface of the movable top board 1a is a horizontal plane, and the direction perpendicular to the placement surface of the movable top board 1a is the vertical direction. Therefore, when imaging the subject M in a recumbent posture, the movable top board 1a is translated in the vertical direction, which is perpendicular to the placement surface of the movable top board 1a. Note that the movable top board 1a may be configured to perform a tilt operation (inclination) by rotating about the axis (y-axis in the figure) in the lateral direction of the movable top board 1a.

The movable top board 1a and the multi-joint arms 2 and 3 are moved as described above, and an X-ray detection signal obtained by the X-ray detector 31 detecting the X-rays irradiated from the X-ray tube 21 is processed by the image processing unit 41 (see FIG. 3) to obtain an X-ray image of the subject M. In the case of performing a fluoroscopic inspection, a plurality of X-ray images is sequentially obtained by irradiating X-rays with a lesser dose than in X-ray imaging from the X-ray tube 21, and each X-ray image is displayed on a monitor 48 (see FIG. 3) in real time. In the case of performing X-ray imaging, X-rays are irradiated from the X-ray tube 21, and a single X-ray image is output to the monitor 48 to be displayed or output to a printer (not shown) to be printed out.

The controller 42 (see FIG. 3) collectively controls each configuration of the X-ray imaging apparatus. In particular, the controller 42 controls the movable top board 1a and the multi-joint arms 2 and 3, and also controls to notify an operator via the monitor 48 that the SSD at the time of imaging measured by the distance sensor 43 (see FIG. 3) falls below the reference SSD in which imaging is allowed when the SSD falls below the reference SSD. In this Example 1, the controller makes the aforementioned notification and simultaneously controls the multi-joint arm 2 for an X-ray tube so as to stop the rotation operation of the X-ray tube 21. In FIG. 3, for the sake of convenience of illustration, wiring connecting the controller 42 to the configuration controlled by the controller 42 is omitted except for a part thereof. The controller 42 corresponds to the control means in the present invention.

The image processing unit 41 and the controller 42 are composed of a central processing unit (CPU) or the like. Note that the image processing unit 41 may be configured by a GPU (Graphics Processing Unit) or the like.

The distance sensor 43 (see FIG. 3) is attached to the X-ray tube 21 and measures the SSD, which is the distance between the focal point of the X-ray tube 21 and the surface (skin) of the subject M. The distance sensor 43 is composed of, for example, an optical sensor. As shown in FIG. 1 and FIG. 2, when the X-ray tube 21 is positioned below the movable top board 1a, the distance sensor 43 measures the distance between the focal point of the X-ray tube 21 and the movable top board 1a. Since the thickness of the movable top board 1a is known, the SSD can be obtained by geometrical calculations using the thickness of the movable top board 1a, the irradiation angle of the X-rays at the time of imaging, and the distance to the measured movable top board 1a. When the X-ray tube 21 is positioned above the movable top board 1a, the distance sensor 43 can directly measure the SSD. Note that the distance sensor 43 corresponds to the SSD deriving means in the present invention.

The comparator 44 (see FIG. 3) compares the SSD at the time of imaging measured by the distance sensor 43 and the reference SSD in which imaging is allowed which has been previously set by input unit 46 (see FIG. 3) and stored in the memory unit 46 (see FIG. 3). The comparator 44 is composed of a comparator, such as, e.g., an operational amplifier. Note that the comparator 44 corresponds to the comparing means in the present invention.

A high voltage generator 45 (see FIG. 3) generates a high voltage for the X-ray tube 21. The settings of the tube current and the tube voltage of the X-ray tube 21 are performed by the high voltage generator 45 based on a command from the controller 42. Note that the product of the tube current value [mA] and the pulse width (exposure time) [s] of the X-ray irradiation is also called “mAs value” and is proportional to the dose (area dose).

The input unit 46 (see FIG. 3) sends data and instructions entered by an operator to the controller 42. The input unit 46 is composed of a pointing device typified by a mouse, a keyboard, a joystick, a trackball, a touch panel, or the like. In Example 1, the reference SSD in which imaging is allowed is set by inputting with the input unit 46 as a reference physical quantity associated with the incident dose in which imaging is allowed, which is also applied to later-described Examples 2 to 6. Note that the input unit 46 corresponds to the setting means in the present invention.

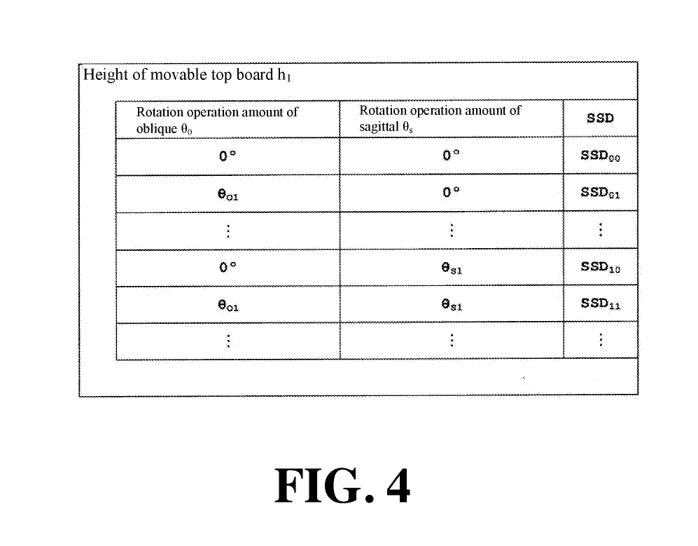

The memory unit 47 (see FIG. 3) writes and stores, via the controller 42, data, such as, e.g., a combination (see FIG. 4) of the X-ray image obtained by the image processing unit 41, the rotation operation amount (rotation operation amount of oblique) of the X-ray tube 21 about the body axis of the subject M, the rotation operation amount (rotation operation amount of sagittal) of the X-ray tube 21 in the body axis direction of the subject M and the operation amount of the movable top board 1a in the vertical direction perpendicular to the placement surface of the movable top board 1a), which does not fall below the reference SSD in which imaging is allowed, reads out the data as the need arises, and sends each date to the monitor 48 via the controller 42 to be displayed. Further, in this Example 1, the memory unit writes and stores the reference SSD in which imaging is allowed, which is input by the input unit 46, reads out at the time of imaging, and sends the reference SSD in which imaging is allowed to the comparator 44 via the controller 42, which is also applied to Examples 2 to 6 which will be described later. The memory unit 47 is composed of a storage medium typified by a ROM (Read-only Memory), a RAM (Random Access Memory), and the like. Note that the memory unit 47 corresponds to the storage means in the present invention.

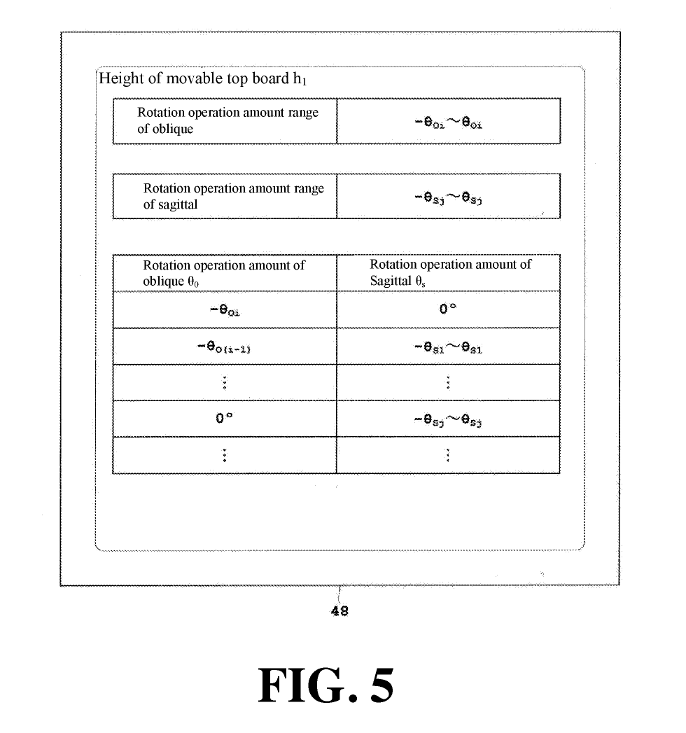

The monitor 48 (see FIG. 3) is configured to display the X-ray image obtained by the image processing unit 41 in real time when fluoroscopic inspection is performed, and read out the X-ray image obtained by the image processing unit 41 and stored in the memory unit 47 as the needs arises to display the X-ray image when imaging is performed. The monitor 48 is configured to read out and display the aforementioned combination stored in the memory unit 47 according to the height of the movable top board 1a (see FIG. 5). Further, the monitor 48 is configured to display a screen (for example, an error message or a marker or a color indicating a warning) notifying an operator of that the SSD at the time of imaging measured by the distance sensor 43 falls below the reference SSD in which imaging is allowed when the SSD at the time of imaging measured with the distance sensor 43 falls below the reference SSD in which imaging is allowed. Note that the monitor 48 corresponds to the display means in the present invention.

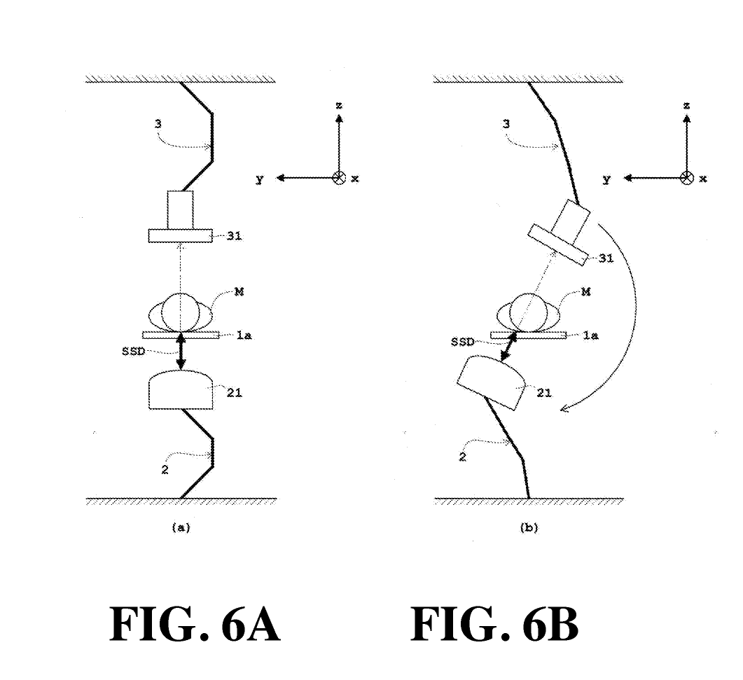

Next, a specific control in Example 1 will be described with reference to FIG. 4 to FIG. 6. FIG. 4 is a schematic diagram showing a combination of a rotation operation amount of oblique, a rotation operation amount of sagittal, and an operation amount of the movable top board in the vertical direction, which do not fall below the reference SSD in which imaging is allowed. FIG. 5 is a display mode of a monitor showing a range of a rotation operation amount of oblique and a range of a rotation operation amount of sagittal at a certain height of the movable top board, which do not fall below the reference SSD in which imaging is allowed. FIG. 6 is a front view of the X-ray tube and the X-ray detector showing a change of an SSD by an oblique rotation, which is a moving operation of the X-ray tube according to Example 1.

As described above, the subject M (see FIG. 1 and FIG. 2) is not a spherical shape but a plate-like shape, so the SSD becomes smaller when both the sagittal and the oblique become deeper with reference to the irradiation from immediately above or immediately below. As shown in FIG. 1 and FIG. 2, when the X-ray tube 21 is positioned below the movable top board 1a, the irradiation angle is set to 0° with reference to the irradiation from immediately below by the X-ray tube 21. The rotation operation amount when rotating the X-ray tube 21 from the irradiation angle 0° with reference to the irradiation from immediately below and the SSD at that time are associated with each other in advance, and written in the memory unit 47 (see FIG. 3) and stored.

For each height of the flexible top board 1a (see FIG. 1 to FIG. 3), within the range that does not fall below the reference SSD in which imaging is allowed, as shown in FIG. 4, the rotation operation amount when rotating the X-ray tube 21 from the irradiation angle 0° and the SSD at that time are associated with each other in advance, and written in the memory unit 47 and stored. Then, the range of the rotation operation amount of oblique and the range of rotation operation amount of sagittal at the height of the movable top board 1a at the time of imaging, which do not fall below the reference SSD in which imaging is allowed are read out from the memory unit 47 and displayed on the monitor 48 (see FIG. 3). One mode at that time is shown in FIG. 5.

In order to obtain the SSD in the combination shown in FIG. 4, the SSD is measured by the distance sensor 43 (see FIG. 3) for measuring the aforementioned SSD. Note that the SSD may be calculated by calculations without using a distance sensor. For example, the SSD may be calculated from the relative position between the shape data of the subject M captured in advance and the focal point of the X-ray tube 21. Further, the SSD may be calculated from a relative position between a model (a model which is imitated by an oval spherical shape imitating a head of a subject M or a plate-like shape imitating a middle of a subject M) imitating the subject M and the focal point of the X-ray tube 21. In the case of calculating the SSD by calculations, the controller 42 (see FIG. 3) performs the calculations. Therefore, in the case of calculating the SSD by calculations, the controller 42 corresponds to the SSD deriving means in the present invention.

In FIG. 5, the range of the rotation operation amount of oblique (−θ_{Oi} to θ_{Oi} in FIG. 5) at the height (h₁ in FIG. 5) of the movable top board 1a at the time of imaging when the rotation of sagittal is stopped and the range of the rotation operation amount of sagittal (−θ_{Sj} to θ_{Sj} in FIG. 5) at the height (h₁) of the flexible top board 1a in at the time of imaging when the rotation of oblique is stopped, in which the ranges does not fall below the reference SSD in which imaging is allowed, are displayed on the monitor 48. Further, in FIG. 5, the range of the rotation operation amount θₒ of oblique at the height (h₁) of the movable top board 1a and the range of the rotation operation amount θ_{S} of sagittal, which do not fall below the reference SSD in which imaging is allowed, are displayed on the monitor 48.

For example, when the rotation operation amount θ_{O} of oblique is −θ_{Oi} which is the lower limit range, the range of the rotation operation amount θ_{S} of sagittal at the height (h₁) of the movable top board 1a which does not fall below the reference SSD in which imaging is allowed is 0°. On the other hand, when the rotation operation amount θ₀ of oblique is −_{θ(i-1)} (−θ_{Oi}<−θ_{θ(i-1)}), the range of the rotation operation amount θ_{S} of sagittal at the height (h₁) of the movable top board 1a which does not fall below the reference SSD in which imaging is allowed is −θ_{S1} to θ_{S1}. In the same manner, the ranges of the rotation operation amount θ_{S} of sagittal at the height (h₁) of the movable top board 1a when the rotation operation amount θ₀ of oblique is at a predetermined angle, which does not fall below the reference SSD in which imaging is allowed, are each displayed on the monitor 48.

Note that in FIG. 6, the illustration of the main body 1b shown in FIG. 1 and FIG. 2 is omitted. The X-ray tube 21 is obliquely rotated with reference to the irradiation from immediately below by the X-ray tube 21 shown in FIG. 6A. In synchronization with the oblique rotation of the X-ray tube 21, the controller 42 controls so that the X-ray detector 31 obliquely rotates and the X-ray tube 21 and the X-ray detector 31 face via the movable top board 1a. As a result, the SSD becomes smaller as shown in FIG. 6B.

The comparator 44 (see FIG. 3) compares the SSD at the time of imaging measured with the distance sensor 43 (see FIG. 3) and the reference SSD in which imaging is allowed. Using the comparison result by the comparator 44, the controller 42 notifies that the SSD at the time of imaging falls below the reference SSD when the SSD falls below the reference SSD, and simultaneously performs the moving operation (stop of the rotation operation of the X-ray tube 21 in this Example 1) of the X-ray tube 21 so that the SSD at the time of imaging does not fall below the reference SSD.

In this Example 1, the controller 42 controls so as to notify an operator via the monitor 48 that the SSD at the time of imaging measured by the distance sensor 43 falls below the reference SSD in which imaging is the allowed when the SSD at the time of imaging measured by the distance sensor 43 falls below the reference SSD in which imaging is the allowed. For example, the notification to the operator is performed by displaying a screen (such as, e.g., an error message, a marker or a color indicating warning) to be notified to the operation on the monitor 48 (see FIG. 3). Note that the controller may control so that the operator is notified by a means other than a monitor. For example, the controller may control to notify the operator with a warning sound by a buzzer (not shown). Further, in Example 1, the controller 42 makes the aforementioned notification and simultaneously controls the multi-joint arm 2 for the X-ray tube so as to stop the oblique rotation operation of the X-ray tube 21. In FIG. 6, the control in the change of the SSD by the oblique rotation is described as an example, but the control in the change of the SSD by the sagittal rotation is similar, so the explanation is omitted.

The X-ray imaging apparatus according to Example 1 is provided with an SSD deriving means (distance sensor 43 in each Example) for obtaining the SSD which is a distance between the focal point of the X-ray tube 21 and the surface of the subject M. Further, the X-ray imaging apparatus is further provided with setting means (input unit 46 in each Example) for setting the reference physical quantity (reference SSD in Examples 1 to 6) associated with the incident dose in which imaging is allowed, and comparing means (comparator 44 in each Example) for comparing the physical quantity (SSD at the time of imaging in Examples 1 to 6) at the time of imaging associated with the incident dose obtained by using the SSD obtained by the SSD deriving means (distance sensor 43) and the reference physical quantity (reference SSD) set by the setting means (input unit 46). By providing such setting means (input unit 46) and comparing means (comparator 44), it can be determined by the comparing means (comparator 44) whether or not the physical quantity (SSD at the time of imaging) at the time of imaging associated with the incident dose obtained by using the SSD falls within the imaging allowable range with reference to the physical quantity (reference SSD). By providing the control means (controller 42 in each Example) for performing predetermined operations (notification and stop of the rotation operation of the X-ray tube 21 in this Example 1) using the comparison result by the comparing means (comparator 44), the incident dose can be suppressed by carrying out the specified operations (notification and stop of the rotation operation of the X-ray tube 21).

As the aforementioned predetermined operations, the following operations can be exemplified. The control means performs (controller 42) the movement operation of the X-ray tube 21 so that the physical quantity (SSD at the time of imaging) falls within the imaging allowable range with reference to the reference physical quantity (reference SSD) by using the comparison result by the comparing means (comparator 44). Further, the control means (controller 42) notifies that the physical quantity (SSD at the time of imaging) at the time of imaging deviates the imaging allowable range with reference to the reference physical quantity (reference SSD) when the physical quantity (SSD at the time of imaging) at the time of imaging deviates the imaging allowable range with reference to the reference physical quantity (reference SSD) by using the comparison result by the comparing means (comparator 44). In this Example 1, this notification and the moving operation of the X-ray tube 21 are combined.

The physical quantity (associated with incident dose), which becomes a target of the comparison, is the aforementioned SSD in this Example 1, which is also applied to Examples 2 to 6 to be described later. When the physical quantity is the SSD, the comparing means (comparator 44) compares the SSD at the time of imaging and the reference SSD in which imaging is allowed, and the control means (controller 42) performs the moving operation of the X-ray tube 21 such that the SSD at the time of imaging does not fall below the reference SSD by using the comparison result by the comparing means (comparator 44). When the SSD at the time of imaging falls below the reference SSD, the SSD becomes too small and therefore the incident dose deviates from the imaging allowable range and increases. Therefore, by performing the moving operation of the X-ray tube 21 such that the SSD at the time of imaging does not fall below the reference SSD, the SSD can be kept large so that the incident falls within the imaging allowable range, which can suppress the incident dose. The aforementioned SSD deriving means is the distance sensor 43 for measuring the SSD in each Example.

A specific mode of the notification in this Example 1 will be described. In cases where the physical quantity (associated with the incident dose) is the aforementioned SSD like in Example 1, the comparing means (comparator 44) compares the SSD at the time of imaging and the reference SSD in which imaging is allowed, and the control means (the controller 42) makes the notification that the SSD at the time of imaging falls below the reference SSD when the SSD at the time of imaging falls below the reference SSD and simultaneously performs the moving operation of the X-ray tube 21 so that the SSD at the time of imaging does not fall below the reference SSD by using the comparison result by the comparing means (controller 42). The above is also applied to Examples 2 to 6 to be described later. It is possible to automatically notify a user (operator) that the SSD at the time of imaging falls below the reference SSD and perform the moving operation of the X-ray tube 21 so that the SSD at the time of imaging does not fall below the reference SSD.

As the moving operation of the X-ray tube 21, the rotation operation of the X-ray tube 21 is primarily exemplified. That is, in this Example 1, the control means (controller 42) makes the aforementioned notification and simultaneously stops the rotation operation of the X-ray tube 21. With this, the rotation operation is prevented from reaching a deep angle with reference to the irradiation immediately above or immediately below. As described above, since the subject M does not have a spherical but has a plate-like shape, when both the sagittal and the oblique with reference to the irradiation immediately above or immediately below reaches a deep depth, the SSD becomes small. However, by stopping the rotation operation of the X-ray tube 21 simultaneously with the aforementioned notification, the SSD at the time of imaging keeps the reference SSD so as not to rotate at a deep angle.

### Example 2

Next, Example 2 of the present invention will be described with reference to the attached drawings.

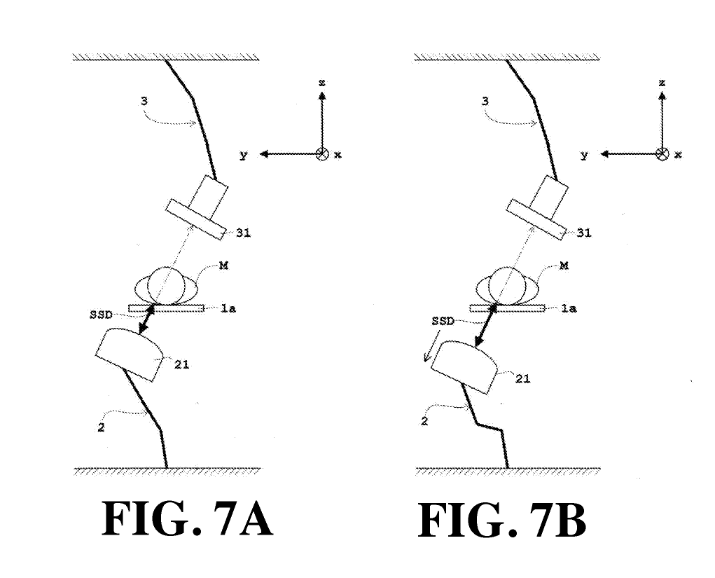

FIG. 7 is a front view of the X-ray tube and the X-ray detector showing the change of the SSD by the oblique rotation, which is a moving operation of the X-ray tube according to Example 2. As for the configuration common to Example 1, the same reference numeral is allotted, and the detailed description thereof is omitted. Note that, in Example 2, the X-ray imaging apparatus shown in FIGS. 1 and 2 which is the same as that of Example 1 described above is used, which is also applied to Examples 3 to 7 to be described later.

In Example 1 described above, as the moving operation of the X-ray tube 21, the rotation operation of the X-ray tube 21 is stopped. On the other hand, in this Example 2, as the moving operation of the X-ray tube 21, as shown in FIG. 7, the X-ray tube 21 is retracted so as to move the X-ray tube 21 away from the subject M. In the same manner as in the case shown in FIG. 6 of Example 1 described above, the explanation will be made taking the oblique rotation as an example.

In FIG. 7, in the same manner as in FIG. 6 of Example 1 described above, the illustration of the main body 1b shown in FIG. 1 and FIG. 2 is omitted. Specifically, it is assumed that the X-ray tube 21 is obliquely rotated with reference to the irradiation from immediately below by the X-ray tube 21 shown in FIG. 6A, and as shown in FIG. 7A, the SSD decreases to the reference SSD. In that case, as shown in FIG. 7B, the X-ray tube 21 is retracted so as to move the X-ray tube 21 away from the subject M.

In this way, in FIG. 7, by retracting the X-ray tube 21 so as to move the X-ray tube 21 away from the subject M, the SSD at the time of imaging increases, and therefore a margin can be obtained. However, in the case of continuously performing the rotation operation, when the rotation operation amount is large, there is a possibility that the rotation center of the X-ray tube 21 and that of the X-ray detector 31 are displaced, which causes the imaging site to deviate the field of view. Therefore, it is preferable to fix the rotation center by retracting the X-ray detector 31 away from the subject M by the amount corresponding to the retract movement of the X-ray tube 21 so that the rotation center and the imaging site coincide with each other. Needless to say, in the case of continuously performing the rotational operation, when the rotation operation amount is small and therefore the imaging site does not deviate the field of view even if the rotation centers are shifted, it is not always necessary to match the rotation center and the imaging site.

The comparator 44 (see FIG. 3) compares the SSD at the time of imaging measured with the distance sensor 43 (see FIG. 3) and the reference SSD in which imaging is allowed. Using the comparison result by the comparator 44, the controller 42 (see FIG. 3) notifies that the SSD at the time of imaging falls below the reference SSD when the SSD falls below the reference SSD, and simultaneously performs the moving operation (stop of the rotation operation of the X-ray tube 21 in this Example 2) of the X-ray tube 21 so that the SSD at the time of imaging does not fall below the reference SSD. In FIG. 7, the description is made by exemplifying the control in the change of the SSD by the oblique rotation, but the control in the change of the SSD by sagittal is similar, so the explanation is omitted.

The X-ray imaging apparatus according to this Example 2 is provided with SSD deriving means (distance sensor 43 in each Example) for deriving the SSD which is a distance between the focal point of the X-ray tube 21 and the surface of the subject M, setting means (input unit 46 in each Example) for setting the reference physical quantity amount (the reference SSD in Examples 1 to 6) associated with the incident dose in which imaging is allowed, and comparing means (comparator 44 in each Example) for comparing the physical quantity (the SSD at the time of imaging in Examples 1 to 6) at the time of imaging associated with the incident dose obtained by using the SSD obtained by the SSD deriving means (the distance sensor 43) and the reference physical quantity (the reference SSD) set by the setting means (input unit 46). By providing them, it can be determined by the comparing means (the comparator 44) whether or not the physical quantity (the SSD at the time of imaging) at the time of imaging associated with the incident dose obtained by using the SSD falls within the imaging allowable range with reference to the physical quantity (the reference SSD). By providing the control means (the controller 42 in each Example) for performing predetermined operations (the notification and the retraction movement of the X-ray tube 21 in this Example 2) using the comparison result by the comparing means (the comparator 44), the incident dose can be suppressed by carrying out the specified operations (the notification and the retraction movement of the X-ray tube 21).

In the same manner as in Example 1 described above, in Example 2, the control means (controller 42) notifies that the physical quantity (SSD at the time of imaging) at the time of imaging deviates the imaging allowable range with reference to the reference physical quantity (reference SSD) when the physical quantity (SSD at the time of imaging) at the time of imaging deviates the imaging allowable range with reference to the reference physical quantity (reference SSD) by using the comparison result by the comparing means (comparator 44). In the same manner as in Example 1 described above, in Example 2, this notification and the moving operation of the X-ray tube 21 are combined.

In the same manner as in Example 1 described above, the physical quantity (associated with the incident dose), which becomes a target of the comparison, is the aforementioned SSD in this Example 2. When the SSD at the time of imaging falls below the reference SSD, the SSD becomes too small and therefore the incident dose deviates from the imaging allowable range and increases. Therefore, by performing the moving operation of the X-ray tube 21 such that the SSD at the time of imaging does not fall below the reference SSD, the SSD can be kept large so that the incident dose falls within the imaging allowable range, which can suppress the incident dose.

In the same manner as in Example 1 described above, in Example 2, the control means (controller 42) notifies that the SSD at the time of imaging falls below the reference SSD when the SSD falls below the reference SSD, and simultaneously performs the moving operation of the X-ray tube 21 so that the SSD at the time of imaging does not fall below the reference SSD. It is possible to automatically notify a user (operator) that the SSD at the time of imaging falls below the reference SSD and perform the moving operation of the X-ray tube 21 so that the SSD at the time of imaging does not fall below the reference SSD.

In this Example 2, the control means (controller 42) makes the aforementioned notification and simultaneously performs the moving operation of the X-ray tube 21 by retracting the X-ray tube 21 so that the X-ray tube 21 is moved away from the subject M. By retracting the X-ray tube 21, the SSD at the time of imaging increases to give a margin with respect to the reference SSD. Therefore, it becomes possible to continuously perform the operation of the X-ray tube 21 (for example, rotation operation of the X-ray tube 21 in the same direction as the direction immediately before the notification) while keeping the lower limit of the reference SSD.

### Example 3

Next, Example 3 of the present invention will be described with reference to the attached drawings.

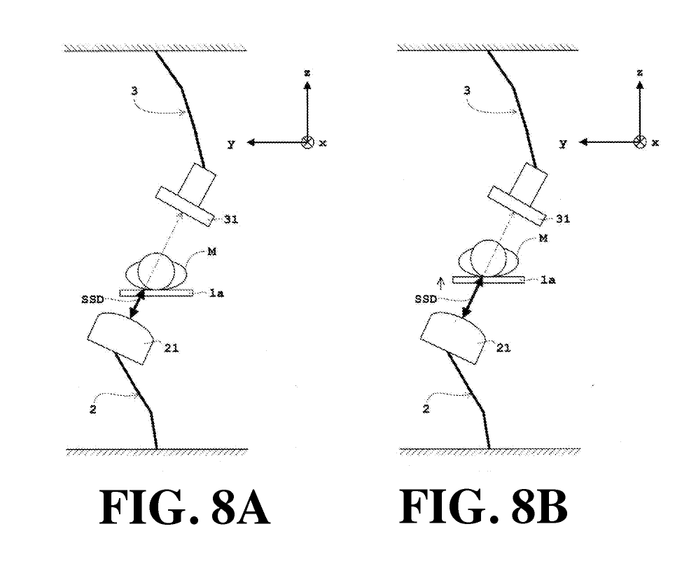

FIG. 8 is a front view of an X-ray tube and an X-ray detector showing the moving operation of the movable top board according to Example 3 and the change of SSD by the oblique rotation. As for the configuration common to Examples 1 and 2, the same reference numeral is allotted, and the detailed description thereof will be omitted. Note that, in this Example 3, the X-ray imaging apparatus shown in FIG. 1 and FIG. 2 which is the same as that of Examples 1 and 2 described above is used, which is also applied to Examples 4 to 7 to be described later.

In the aforementioned Examples 1 and 2, it is notified that the SSD at the time of imaging falls below the reference SSD when the SSD falls below the reference SSD, and simultaneously the moving operation (stop of the rotation operation of the X-ray tube 21 in Example 1 described above, retraction of the X-ray tube 21 in Example 2 described above) of the X-ray tube 21 was performed so that the SSD of at the time of imaging does not fall below the reference SSD. On the other hand, in this Example 3, as the predetermined operation, as shown in FIG. 8, the movable top board 1a is translated in a direction perpendicular to the placement surface of the movable top board 1a so that the subject M moves away from the X-ray tube 21. In the same manner as in the case shown in FIG. 6 of Example 1 described above and the case shown in FIG. 7 of Example 2 described above, the explanation will be made taking the oblique rotation as an example.

In FIG. 8, in the same manner as in FIG. 6 of Example 1 described above and FIG. 7 of Example 2, the illustration of the main body 1b shown in FIG. 1 and FIG. 2 will be omitted. Specifically, it is assumed that the X-ray tube 21 is obliquely rotated with reference to the irradiation from immediately below by the X-ray tube 21 shown in FIG. 6A, and as shown in FIG. 8A, the SSD decreases to the reference SSD. In such a case, as shown in FIG. 8B, the movable top board 1a is translated in a direction perpendicular to the placement surface of the movable top board 1a so that the subject M is moved away from the X-ray tube 21.

As shown in FIG. 8, when the X-ray tube 21 is positioned below the movable top board 1a and the subject M is in a recumbent posture, the placement surface of the movable top board 1a is a horizontal plane. Therefore, by moving the movable top board 1a upward in the vertical direction which is perpendicular to the placement surface of the movable top board 1a, the subject top M is moved away from the X-ray tube 21. To the contrary, when the X-ray tube 21 is positioned above the movable top board 1a and the subject M in a recumbent posture is imaged, by downwardly moving the movable top board 1a in the vertical direction which is perpendicular to the placement surface of the movable top board 1a, the subject top M is moved away from the X-ray tube 21.

In this way, in FIG. 8, by moving the movable top board 1a upward to move the subject M away from the X-ray tube 21, the SSD at the time of imaging increases and a margin is obtained. However, in the case of continuously performing the rotation operation, when the rotation operation amount is large, there is a possibility that the rotation center of the X-ray tube 21 and that of the X-ray detector 31 are displaced, which causes the imaging site to fall outside the field of view. Therefore, it is preferable to move the rotation center by the amount that the movable top board 1a is moved upward so that the rotation center and the imaging site are made to coincide. Needless to say, as described in the aforementioned Example 2, in the case of continuously performing the rotational operation, when the rotation operation amount is small and therefore the imaging site does deviate the field of view even if the rotation center deviates, it is not always necessary to match the rotation center and the imaging site.

The comparator 44 (see FIG. 3) compares the SSD at the time of imaging measured with the distance sensor 43 (see FIG. 3) and the reference SSD in which imaging is allowed. Then, using the comparison result by the comparator 44, the controller 42 (see FIG. 3) notifies that the SSD at the time of imaging falls below the reference SSD when the SSD at the time of imaging falls below the reference SSD and simultaneously moves the subject M away from the X-ray tube 21 by moving the movable top board 1a upward in the vertical direction which is perpendicular to the placement surface of the movable top board 1a. In FIG. 8, the control in the change of the SSD by the oblique rotation is described as an example, but the control in the change of the SSD by the sagittal rotation is similar, so the explanation is omitted.

According to the X-ray imaging apparatus according to this Example 3, in the same manner as the X-ray imaging apparatus according to Examples 1 and 2 described above, the X-ray imaging apparatus is provided with SSD deriving means (distance sensor 43 in each Example) for deriving the SSD which is a distance between the focal point of the X-ray tube 21 and the surface of the subject M, setting means (input unit 46 in each Example) for setting the reference physical quantity amount (the reference SSD in Examples 1 to 6) associated with the incident dose in which imaging is allowed, and comparing means (comparator 44 in each Example) for comparing the physical quantity (SSD at the time of imaging in Examples 1 to 6) at the time of imaging associated with the incident dose obtained by using the SSD obtained by the SSD deriving means (distance sensor 43) and the reference physical quantity (reference SSD) set by the setting means (input unit 46). By providing them, it can be determined by the comparing means (comparator 44) whether or not the physical quantity (SSD at the time of imaging) at the time of imaging associated with the incident dose obtained by using the SSD falls within the imaging allowable range with reference to the physical quantity (reference SSD). By providing the control means (controller 42 in each Example) for performing predetermined operations (notification and upward movement of the movable top board 1a in this Example 3) using the comparison result by the comparing means (comparator 44), the incident dose can be suppressed by carrying out the specified operations (notification and upward movement of the movable top board 1a).

In the same manner as in Examples 1 and 2 described above, in Example 3, using the comparison result by the comparing means (comparator 44), the control means (controller 42) notifies that the physical quantity (SSD at the time of imaging) at the time of imaging deviates the imaging allowable range with reference to the reference physical quantity (reference SSD) when the physical quantity (SSD at the time of imaging) at the time of imaging deviates the imaging allowable range with reference to the reference physical quantity (reference SSD). In this Example 3, this notification and the upward movement of the movable top board 1a are combined.

In the same manner as in Examples 1 and 2 described above, in Example 3, the control means (controller 42) notifies that the SSD at the time of imaging falls below the reference SSD when the SSD falls below the reference SSD, and simultaneously performs the moving operation of the X-ray tube 21 so that the SSD at the time of imaging does not fall below the reference SSD. It is possible to automatically notify a user (operator) that the SSD at the time of imaging falls below the reference SSD and perform the moving operation so that the SSD at the time of imaging does not fall below the reference SSD.

In this Example 3, it is controlled to operate the top board (movable top board 1a in each Example) for placing a subject M thereon. That is, the control means (controller 42) makes the notification and simultaneously translate (as shown in FIG. 8 of this Example 3, when the placement surface is horizontal and the X-ray tube is positioned below the top board, the top board 1a is moved upward, and when the placement surface is horizontal and the X-ray tube 21 is positioned above the top board, the top board is moved downward) the top board in a direction perpendicular to the placement surface of the top board so as to move the object M away from the X-ray tube 21 to continuously perform the rotation operation (oblique rotation in the case of FIG. 8) of the X-ray tube 21 in the same direction as a direction immediately before the notification. In the same manner as in the case of retracting the X-ray tube 21 so that the X-ray tube 21 is moved away from the subject M as in Example 2 described above, by translating (upward movement in FIG. 8) the top board (movable top board 1a) in a direction (vertical direction in FIG. 8) perpendicular to the placement surface (horizontal plane in FIG. 8) of the top board (movable top board 1a), the SSD at the time of imaging increases. Thus, a margin is given with respect to the reference SSD, which makes it possible to continuously perform the rotation operation (oblique rotation in FIG. 8) of the X-ray tube 21 in the same direction as the direction immediately before the notification while keeping the lower limit of the reference SSD.

### Example 4

Next, Example 4 of the present invention will be described with reference to the attached drawings.

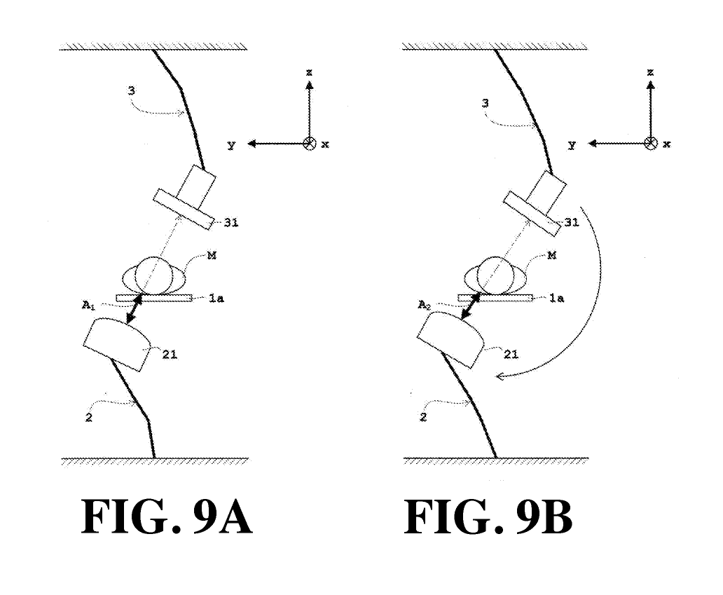

FIG. 9 is a front view of the X-ray tube and the X-ray detector showing a change of an SSD by an oblique rotation, which is a moving operation of the X-ray tube according to Example 4. As for the configuration common to Examples 1 to 3, the same reference numeral is allotted, and the detailed description thereof will be omitted. Note that, in this Example 4, the X-ray imaging apparatus shown in FIG. 1 and FIG. 2 which is the same as that of Examples 1 to 3 described above is used, which is also applied to Examples 5 to 7 to be described later.

In Examples 1 to 3 described above, by setting only one reference SSD, particularly in Example 1, at the time when the SSD at the time of imaging falls below the reference SSD, it is notified that the SSD falls below the reference SSD when the SSD falls below the reference SSD, and simultaneously the rotation operation of the X-ray tube 21 is stopped. On the other hand, in this Example 4, the reference SSD is set in descending order in a plurality of stages, so that the lower limit setting of the reference SSD is reset lower, and the rotation operation of the X-ray tube 21 is continued. In the same manner as in the case shown in FIG. 6 of Example 1 described above, in the case shown in FIG. 7 of Example 2 described above, and the case shown in FIG. 8 of Example 3 described above, the explanation will be made taking the oblique rotation as an example.

In FIG. 9, in the same manner as in FIG. 6 of Example 1 described above, FIG. 7 of Example 2, and FIG. 8 of Example 3, the illustration of the main body 1b shown in FIG. 1 to FIG. 3 will be omitted. Let the value of the reference SSD be A₁>A₂. Specifically, it is assumed that the X-ray tube 21 is obliquely rotated with reference to the irradiation from immediately below by the X-ray tube 21 shown in FIG. 6A, and as shown in FIG. 9A, the SSD decreases to the reference SSD(A₁). In that case, as shown in FIG. 9B, the X-ray tube 21 is obliquely rotated so that the SSD reaches the reference SSD(A₂). In the same manner as in Example 1 described above, in synchronism with the oblique rotation of the X-ray tube 21, the controller 42 (see FIG. 3) controls so that the X-ray detector 31 obliquely rotates and the X-ray tube 21 and the X-ray detector 31 face via the movable top board 1a. As a result, the SSD becomes smaller to the second reference SSD(A₂) as shown in FIG. 9B.

The comparator 44 (see FIG. 3) compares the SSD at the time of imaging measured with the distance sensor 43 (see FIG. 3) and the reference SSD in which imaging is allowed. Then, using the comparison result by the comparator 44, the controller 42 (see FIG. 3) notifies the fact that the SSD at the time of imaging falls below the reference SSD (A₁) when the SSD at the time of imaging falls below the reference SSD (A₁), and simultaneously continuously operates the rotation operation of the X-ray tube 21 until the SSD reaches the rest reference SSD(A₂). In FIG. 9, the control in the change of the SSD by the oblique rotation is described as an example, but the control in the change of the SSD by the sagittal rotation is similar, so the explanation is omitted.

According to the X-ray imaging apparatus according this Example 4, in the same manner as the X-ray imaging apparatus according to Examples 1 to 3 described above, the X-ray imaging apparatus is provided with SSD deriving means (distance sensor 43 in each Example) for deriving the SSD which is a distance between the focal point of the X-ray tube 21 and the surface of the subject M, setting means (input unit 46 in each Example) for setting the reference physical quantity amount (the reference SSD in Examples 1 to 6) associated with the incident dose in which imaging is allowed, and comparing means (comparator 44 in each Example) for comparing the physical quantity (the SSD at the time of imaging in Examples 1 to 6) at the time of imaging associated with the incident dose obtained by using the SSD obtained by the SSD deriving means (the distance sensor 43) and the reference physical quantity (the reference SSD) set by the setting means (input unit 46). By providing them, it can be determined by the comparing means (the comparator 44) whether or not the physical quantity (the SSD at the time of imaging) at the time of imaging associated with the incident dose obtained by using the SSD falls within the imaging allowable range with reference to the physical quantity (the reference SSD). By providing the control means (the controller 42 in each Example) for performing predetermined operations (the notification and the suspension of the rotation operation of the X-ray tube 21 in this Example 4) using the comparison result by the comparing means (the comparator 44), the incident dose can be suppressed by carrying out the specified operations (the notification and the suspension of the rotation operation of the X-ray tube 21).

In the same manner as in Examples 1 to 3 described above, in Example 4, he control means (controller 42) notifies that the physical quantity (SSD at the time of imaging) at the time of imaging deviates the imaging allowable range with reference to the reference physical quantity (reference SSD) when the physical quantity (SSD at the time of imaging) at the time of imaging deviates the imaging allowable range with reference to the reference physical quantity (reference SSD) by using the comparison result by the comparing means (comparator 44). In this Example 4, this notification and the moving operation of the X-ray tube 21 are combined.

In the same manner as in Examples 1 to 3 described above, in Example 4, he control means (controller 42) notifies that the SSD at the time of imaging falls below the reference SSD when the SSD falls below the reference SSD, and simultaneously performs the moving operation of the X-ray tube 21 so that the SSD at the time of imaging does not fall below the reference SSD. It is possible to automatically notify a user (operator) that the SSD at the time of imaging falls below the reference SSD and perform the moving operation so that the SSD at the time of imaging does not fall below the reference SSD.

In this Example 4, the setting of the reference SSD by the setting means (input unit 46) is performed in a plurality of stages. In particular, when “n” is an integer of 2 or more, and “k” is an integer satisfying 2≤k≤n, the setting means (input unit 46) sets the values of the reference SSD in descending order in a plurality of stages in A₁, A₂, . . . , Aₖ, . . . , A₍ₙ₋₁₎, An (that is, A₁>A₂> . . . >Aₖ> . . . >A₍ₙ₋₁₎>An). Then, the control means (controller 42) repeats the step of notifying that the SSD at the time of imaging falls below the reference SSD(A₁) when the SSD at the time of imaging falls below the reference SSD(A₁) and simultaneously continuously performing the rotation operation of the tube 21 until the SSD reaches the reference SSD(A₂) reset by the setting means (input unit 46) and thereafter the step of notifying that the SSD at the time of imaging falls below the reference SSD (A₍ₖ₋₁₎) when the SSD at the time of imaging falls below the reference SSD (A₍ₖ₋₁₎) and simultaneously continuously performing the rotation operation of the X-ray tube 21 until the SSD reaches the reference SSD(Aₖ) reset by the setting means (input unit 46). Further, the control means notifies that the SSD at the time of imaging falls below the reference SSD(Aₙ) when the SSD falls below the reference SSD(Aₙ) and simultaneously stops the rotation operation of the X-ray tube 21. In this way, when it is desired to rotate the X-ray tube 21 to a deeper angle with reference to the irradiation from immediately above or immediately below, the reference SSD is set in a descending order in a plurality of stages.

In particular, when n=2, the value of the reference SSD is set to A₁ and A₂ in descending order in two stages (that is, A₁>A₂). The control means (controller 42) makes a notification that the SSD at the time of imaging falls below the reference SSD(A₁) when the SSD at the time of imaging falls below the SSD(A₁) and simultaneously continuously performs the rotation operation of the X-ray tube 21 until the SSD at the time of imaging reaches the reference SSD(A₂) reset by the setting means (input unit 46), and makes a notification that the SSD at the time of imaging falls below the reference SSD(A₂) when the SSD at the time of imaging falls below the SSD(A₂) and simultaneously stops the X-ray tube 21. In this way, when limiting to n=2, in cases where it is desired to rotate the X-ray tube 21 to a deeper angle with reference to the irradiation from immediately above or immediately below, the reference SSD is set in descending order in two steps.

### Example 5

Next, Example 5 of the present invention will be described with reference to the attached drawings.

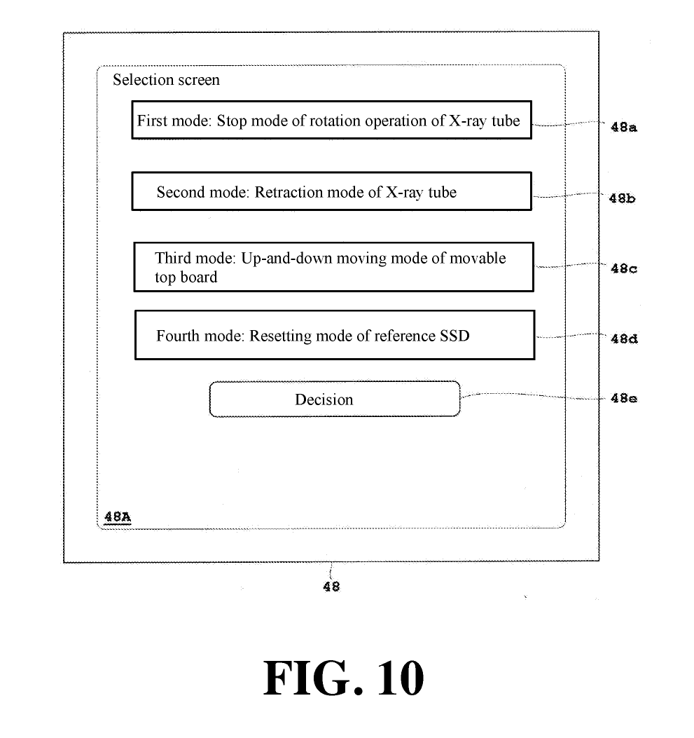

FIG. 10 is a display mode of a monitor selection screen according to Examples 5 and 6. As for the configuration common to Examples 1 to 4, the same reference numeral is allotted, and the detailed description thereof will be omitted. Note that, in this Example 5, the X-ray imaging apparatus shown in FIG. 1 and FIG. 2 which is the same as that of Examples 1 to 4 described above is used, which is also applied to Examples 6 to 7 to be described later.

In Example 5, an input unit 46 (see FIG. 3) is provided, which is also applied to Example 6. The input unit 46 has a function of selection means for selecting one moving operation mode from a plurality of moving operation modes of the X-ray tube 21 (see FIG. 1 and FIG. 2, etc.), the X-ray detector 31 (see FIGS. 1 and 2, etc.), and the movable top board 1a (see FIG. 1 and FIG. 2, etc.). In cases where the monitor is configured by a touch panel and the monitor is also used for the function of the input unit 46, when an operator touches each button on the selection screen of the monitor, any one moving operation mode is selected. In cases where the input unit 46 is configured by a pointing device, when the pointer is clicked in a state in which the pointer points each button on the selection screen of the monitor, any one moving operation mode is selected. Note that the input unit 46 corresponds to the selection means in the present invention.

In the monitor 48 of FIG. 10, there is provided a selection screen 48A for selecting any one moving operation mode from the moving operation modes of Examples 1 to 4 described above. In FIG. 10, the moving operation mode of Example 1 is defined as a first mode (stop mode of the rotation operation of the X-ray tube) 48a, the moving operation mode of Example 2 is defined as a second mode (retraction mode of the X-ray tube) 48b, the moving operation mode of Example 3 is defined as a third mode (up-down-movement of movable top board) 48c, and the moving operation mode of Example 4 is defined as a fourth mode (resetting mode of the reference SSD) 48d. In FIG. 10, the moving operation mode of the X-ray tube 21 and the movable top board 1a are shown, but the mode is not limited to the moving operation mode of Examples 1 to 4 described above, and the moving operation mode of the X-ray detector may be incorporated.

Prior to the operation of the apparatus, the selection screen 48A as shown in FIG. 10 is displayed on the monitor 48. An operator selects one of the moving operation modes from the first to fourth modes 48a to 48d to highlight the moving operation mode, and finally decides and selects by the decision button 48e. After the selection, imaging is performed. When the SSD at the time of imaging falls below the reference SSD, a notification that the SSD falls below the reference SSD is made and simultaneously the moving operation of the X-ray tube 21 or the movable top board 1a is performed according to the selected moving operation mode. As for the method of selecting the moving operation mode, it is not limited to the method shown in FIG. 10. For example, at the time when one of the moving operation modes is selected from the first to fourth modes 48a to 48d, the moving operation mode may be finally selected.

In this Example 5, the control means (controller 42 in each Example) makes the aforementioned notification and simultaneously performs the moving operation of the X-ray tube 21, the X-ray detector 31, or the top board (movable top board 1a in each Example) according to the moving operation mode (one of moving operation mode of the first to fourth modes 48a to 48d in FIG. 10) selected by the aforementioned selection means (input unit 46 in this Example 5). In the case of this Example 5, by performing the selection in advance before the operation of the apparatus, it is possible to make the notification and simultaneously quickly perform the moving operation of the X-ray tube 21, the X-ray detector 31, or the top board (movable top board 1a) according to the moving operation mode selected by the aforementioned selection means (input unit 46), which exerts an effect that the operation becomes simplified.

### Example 6

Next, Example 6 of the present invention will be described with reference to the attached drawings.

As for the configuration common to Examples 1 to 5, the same reference numeral is allotted, and the detailed description thereof will be omitted. Note that, in this Example 6, the X-ray imaging apparatus shown in FIG. 1 and FIG. 2 which is the same as that of Examples 1 to 5 described above is used, which is also applied to Example 7 to be described later. In this Example 6, a predetermined moving operation mode is selected using the monitor shown in FIG. 10 which is the same as the example 5 described above.

In the same manner as in Example 5 described above, in Example 6, an input unit 46 (see FIG. 3) is provided. The input unit 46 has a function of selection means for selecting one moving operation mode from a plurality of moving operation modes of the X-ray tube 21 (see FIG. 1 and FIG. 2, etc.), the X-ray detector 31 (see FIG. 1 and FIG. 2, etc.), and the movable top board 1a (see FIG. 1 and FIG. 2, etc.).

In the same manner as in Example 5 described above, in the monitor 48 of FIG. 10, there is provided a selection screen 48A for selecting any one moving operation mode from the moving operation modes of Examples 1 to 4 described above. In Example 5 described above, by performing the selection in advance before the operation of the apparatus, simultaneously with the notification, the moving operation of the X-ray tube 21, the X-ray detector 31, or the movable top board 1a is performed in accordance with the moving operation mode (one moving operation mode of the first to fourth modes 48a to 48d in FIG. 10) selected by the input unit 46.

On the other hand, in this Example 6, immediately after notifying that the SSD at the time of imaging falls below the reference SSD when the SSD at the time of imaging falls below the reference SSD, the selection screen 48A as shown in FIG. 10 is displayed on the monitor 48. The operator selects any one of the moving operation modes from the first to fourth modes 48a to 48d to highlight the moving operation mode. After the selection by finally deciding by the decision button 48e, the moving operation of the X-ray tube 21 or the movable top board 1a is performed in accordance with the selected moving operation mode.

In Example 6, after making the selection with the aforementioned selection means (input unit 46 in this Example 6) after the aforementioned notification, the control means (controller 42 in each Example) performs the moving operation of the X-ray tube 21, the X-ray detector 31, or the top board (movable top board 1a in each Example) according to the moving operation mode (one of moving operation mode of the first to fourth modes 48a to 48d in FIG. 10) selected by the aforementioned selection means (input unit 46 in this Example 6). In the case of this Example 6, it is advantageous when a user (operator) wishes to select each moving operation mode from time to time after the user (operator) receives the notification.

### Example 7

Next, Example 7 of the present invention will be described with reference to the attached drawings.

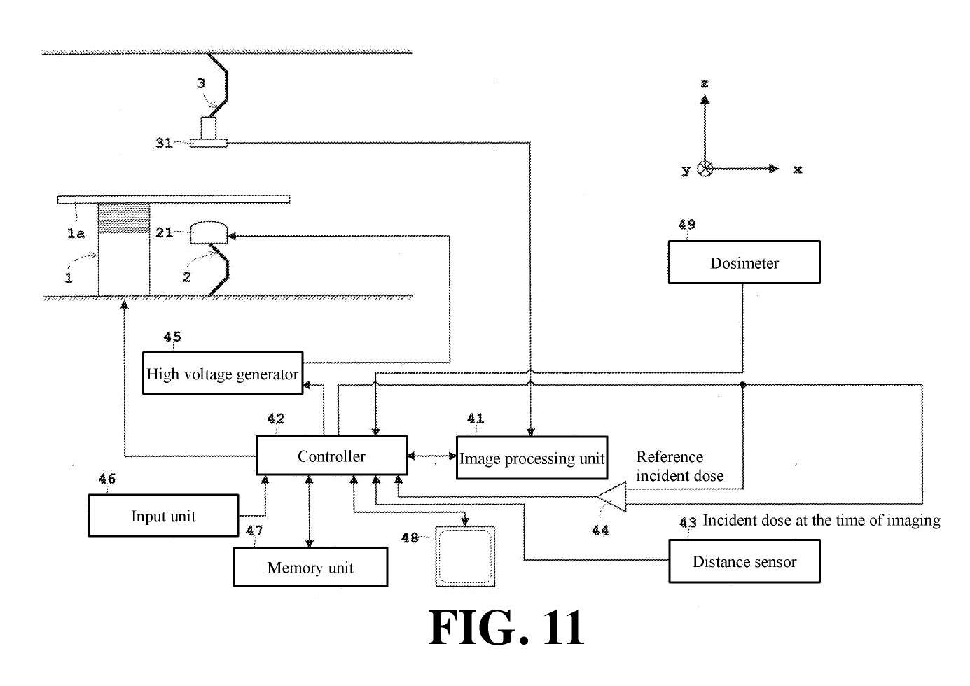

FIG. 11 is a block diagram of an X-ray imaging apparatus according to Example 7 in the case where an incident dose is obtained from an area dose measured with a dosimeter. As for the configuration common to Examples 1 to 6, the same reference numeral is allotted, and the detailed description thereof will be omitted. Note that, in this Example 7, the X-ray imaging apparatus shown in FIG. 1 and FIG. 2 which is the same as that of Examples 1 to 6 described above is used.

In Examples 1 to 6 described above, the physical quantity (associated with the incident dose), which becomes a target of the comparison, is the aforementioned SSD. The comparing means (comparator 44 in each of Examples 1 to 6) compares the SSD at the time of imaging and the reference SSD in which imaging is allowed. Using the comparison result by the comparing means (comparator 44), the control means (controller 42 in each of Examples 1 to 6) performs the moving operation of the X-ray tube 21 or the predetermined operation (notification in each of Examples 1 to 6 and the up-and-down movement of the movable top board 1a in Example 3) such that the SSD at the time of imaging does not fall below the reference SSD. On the other hand, in this Example 7, the physical quantity (associated with the incident dose), which becomes a target of the comparison, is an incident dose. In the case of the incident dose, it can be obtained by multiplying a value obtained by dividing the product of the area dose [Gy·m²] measured by a dosimeter or the tube current value [A] and the imaging time [s] by the square [m²] of the SSD by a factor.

As shown in FIG. 11, when obtaining the incident dose from the area dose [Gy·m²] measured by the dosimeter 49, the incident dose is expressed by the following Equation (1).

D=α×S×(1/SSD²) (1)

D in Equation (1) is an incident dose [Gy], α is a coefficient, and S is an area dose [Gy·m²] output from a dosimeter. That is, according to Equation (1), the incident dose D[Gy] is obtained by multiplying the value obtained by dividing the area dose S[Gy·m²] by the square [m²] of the SSD by the coefficient α. The coefficient α is appropriately set beforehand according to the type of dosimeter. The controller 42 (see FIG. 11) performs the calculation according to the above-mentioned Equation (1).

In this Example 7, a reference incident dose in which imaging is allowed is input by the input unit 46 (see FIG. 11) as the reference physical quantity associated with the incident dose in which imaging is allowed. The reference incident dose in which imaging is allowed input by the input unit 46 is written and stored in the memory unit 47 (see FIG. 11) via the controller 42, and read out at the time of imaging. The reference incident dose in which imaging is allowed is sent to the comparator 44 (see FIG. 11) via the controller 42.

Using the SSD at the time of imaging measured with the distance sensor 43 (see FIG. 11), whether or not the incident dose D[Gy] at the time of imaging obtained by the above Equation (1) falls within the imaging allowable range with reference to the reference incident dose is determined by the comparator 44. Specifically, using the SSD at the time of imaging measured with the distance sensor 43, the comparator 44 compares the incident dose D[Gy] at the time of imaging obtained by the above Equation (1) and the reference incident dose in which the imaging is allowed. Then, using the comparison result by the comparator 44, the controller 42 performs the moving operation (for example, stop of the rotation operation of the X-ray tube 21 or retraction of the X-ray tube 21) of the X-ray tube 21 (see FIG. 11) so that the incident dose D[Gy] at the time of imaging falls within the imaging allowable range with reference to the incident dose or a specified operation (for example, notification and the up-and-down movement of the movable top board 1a). Further, as the predetermined operation, the X-ray condition (imaging condition) may be changed so that the incident dose D[Gy] at the time of imaging does not exceed the reference incident dose. For example, the tube voltage value and the tube current value may be lowered, the pulse width (exposure time) of the X-ray irradiation may be shortened, or the frame rate may be lowered.

In cases where the dosimeter shown in FIG. 11 is not available, the incident dose can be obtained from the product of the tube current value [A] and the imaging time [s]. When the incident dose is obtained from the product of the tube current value [A] and the imaging time [s], the incident dose is expressed by the following Equation (2).

D=β×As×(1/SSD²) (2)

D in Equation (2) is an incident dose [Gy], β is a coefficient, and As is a product of the tube current value [A] and the imaging time [s]. That is, according to the above Equation (2), the indecent dose D[Gy] is obtained by multiplying the value obtained by dividing the product As of the tube current value [A] and the imaging time [s] by the square [m²] of the SSD by the factor β. The coefficient β is appropriately set beforehand according to the type of X-ray tube. The controller 42 performs the calculation according to the above Equation (2).

In the same manner as in the case of obtaining the incident dose from the area dose [Gy·m²] measured with the dosimeter 49, the reference incident dose in which imaging is allowed is input and set by the input unit 46. The reference incident dose in which imaging is allowed input and set by the input unit 46 is written and stored in the memory unit 47 via the controller 42, and read out at the time of imaging. The reference incident dose in which imaging is allowed is sent to the comparator 44 via the controller 42.

Using the SSD at the time of imaging measured with the distance sensor 43, whether or not the incident dose D[Gy] at the time of imaging obtained by the above Equation (2) falls within the imaging allowable range with reference to the reference incident dose is determined by the comparator 44. Specifically, using the SSD at the time of imaging measured with the distance sensor 43, the comparator 44 compares the incident dose D[Gy] at the time of imaging obtained by the above Equation (2) and the reference incident dose in which the imaging is allowed. Then, using the comparison result by the comparator 44, the controller 42 performs the moving operation (for example, stop of the rotation operation of the X-ray tube 21 or the retraction of the X-ray tube 21) of the X-ray tube 21 so that the incident dose D[Gy] at the time of imaging falls within the imaging allowable range with reference to the incident dose or a specified operation (for example, notification and the up-and-down movement of the movable top board 1a). In the same manner as in the case of obtaining the incident dose from the area dose [Gy·m²] measured with the dosimeter 49, as the predetermined operation, the X-ray condition (imaging condition) may be changed so that the incident dose D[Gy] at the time of imaging does not exceed the reference incident dose.

According to the X-ray imaging apparatus according this Example 7, in the same manner as the X-ray imaging apparatus according to Examples 1 to 6 described above, the X-ray imaging apparatus is provided with SSD deriving means (distance sensor 43 in each Example) for deriving the SSD which is a distance between the focal point of the X-ray tube 21 and the surface of the subject M, setting means (input unit 46 in each Example) for setting the reference physical quantity amount (the reference SSD in Example 7) associated with the incident dose in which imaging is allowed, and comparing means (comparator 44 in each Example) for comparing the physical quantity (the incident dose D[Gy] at the time of imaging in Example 7) at the time of imaging associated with the incident dose obtained by using the SSD obtained by the SSD deriving means (the distance sensor 43) and the reference physical quantity (the reference SSD) set by the setting means (input unit 46). By providing them, it can be determined by the comparing means (the comparator 44) whether or not the physical quantity (incident doe D[Gy] at the time of imaging) at the time of imaging associated with the incident dose obtained by using the SSD falls within the imaging allowable range with reference to the reference physical quantity (the reference incident dose). By providing the control means (controller 42 in each Example) for performing predetermined operations (for example, stop of the rotation operation of the X-ray tube 21, retraction of the X-ray tube 21, change of the X-ray condition or up-and-down movement of the movable top board 1a, and notification) by using the comparison result in the comparing means (comparator 44), the incident dose can be suppressed by performing predetermined operations (stop of the rotation operation of the X-ray tube 21, retraction of the X-ray tube 21, change of the X-ray condition or up-and-down movement of the movable top board 1a, and notification).

The present invention is not limited to the aforementioned embodiments, and can be modified as follows.

(1) In each Example described above, the X-ray imaging apparatus is provided with the multi-joint arms 2 and 3 as shown in FIG. 1 and FIG. 2. However, as exemplified by an X-ray imaging apparatus equipped with a holding mechanism (for example, C-arm) for holding the X-ray tube and the X-ray detector and capable of integrally moving the X-ray tube and the X-ray detector by driving the holding mechanism, and the X-ray exposure apparatus capable of integrally moving the X-ray tube and the X-ray detector in conjunction with the movement (for example, a tilt operation) of the top board for placing a subject thereon, the structure to be applied to the X-ray imaging apparatus is not particularly limited.

(2) In each Example described above, as shown in FIG. 1 and FIG. 2, the multi-joint arm 2 for the X-ray tube is supported on the floor surface, and the multi-joint arm 3 for the X-ray detector is suspended from the ceiling surface. However, conversely, it may be applied to the structure in which the multi-joint arm for the X-ray tube is suspended from the ceiling surface and the multi-joint arm 3 of the X-ray detector is supported by a floor surface. That is, in FIGS. 1 and 2, X-rays are irradiated from below, but in cases where the multi-joint arm for the X-ray tube is hung and supported from the ceiling surface, X-rays may be irradiated from above. In particular, when the placement surface of the top board is horizontal and the X-ray tube is positioned below the top board as in Example 3 described above, the top board is translated in a direction perpendicular to the placement surface of the top board so as to move the subject away from the X-ray tube by raising the top board. However, in cases where the multi-joint arm for the X-ray tube is hung and supported from the ceiling surface (the placement surface of the top board is horizontal and the X-ray tube is positioned above the top board), the top board is translated in a direction perpendicular to the placement surface of the top board so that the subject moves away from the X-ray tube by moving the top board downward.

(3) In each Example described above, the SSD deriving means is a distance sensor for measuring the SSD. However, as mentioned in Example 1, the SSD deriving means may also calculate the SSD by calculations. For example, the SSD deriving means may calculate the SSD from the relative position between the shape data of the subject captured in advance and the focal point of the X-ray tube. Further, the SSD deriving means may calculate the SSD from a relative position between a model (a model which is imitated by an oval spherical shape imitating a head of a subject or a plate-like shape imitating a middle of a subject) imitating the subject and the focal point of the X-ray tube.

(4) In each Example described above, it is notified that the physical quantity at the time of imaging (SSD at the time of imaging in Examples 1 to 6, incident dose D[Gy] at the time of imaging in Example 7) deviates from the imaging allowable range with reference to the reference physical quantity (reference SSD in Examples 1 to 6, reference incident dose in Example 7) when the physical quantity at the time of imaging deviates from the imaging allowable range with reference to the reference physical quantity, it is not necessary to make a notification. Only predetermined operations (for example, stop of the rotation operation of the X-ray tube, retraction of the X-ray tube, or rising movement of the top board) may be performed.

### DESCRIPTION OF REFERENCE SYMBOLS

1a: movable top board
21: X-ray tube
31: X-ray detector
42: controller
43: distance sensor
44: comparator
46: input unit
47: memory unit
48: display means
48A: selection screen
49: dosimeter
θ_{O}: rotation operation amount of oblique
θ_{S}: rotation operation amount of sagittal
A₁, A₂, . . . , Aₖ, . . . , A₍ₙ₋₁₎, Aₙ: value of reference SSD
D: incident dose
S: area dose output from the dosimeter
As: product of the tube current value and imaging time
α, β: coefficient
M: subject

1. An X-ray imaging apparatus for performing X-ray imaging, comprising:
an X-ray tube configured to irradiate X-rays;
SSD deriving means configured to derive an SSD, which is a distance between a focal point of the X-ray tube and a surface of a subject;
setting means configured to set a reference physical quantity associated with an incident dose in which imaging is allowed;
comparing means configured to compare a physical quantity at the time of imaging associated with an incident dose derived using the SSD by the SSD deriving means and the reference physical quantity set by the setting means; and
control means configured to perform a predetermined operation using a comparison result by the comparing means.

2. The X-ray imaging apparatus as recited in claim 1, wherein
the control means performs a movement operation of the X-ray tube so that the physical quantity at the time of imaging falls within an imaging allowable range with reference to the reference physical quantity by using the comparison result by the comparing means.

3. The X-ray imaging apparatus as recited in claim 2, wherein
the physical quantity is the SSD, and
the comparing means compares the SSD at the time of imaging and a reference SSD in which imaging is allowed, and
the control means performs a moving operation of the X-ray tube such that the SSD at the time of imaging does not fall below the reference SSD by using the comparison result by the comparing means.

4. The X-ray imaging apparatus as recited in claim 1, wherein
the SSD deriving means is a distance sensor for measuring the SSD.

5. The X-ray imaging apparatus as recited in claim 1, wherein
the SSD deriving means is SSD calculation means that calculate the SSD from a relative position between shape data of the subject imaged in advance and the focal point of the X-ray tube.

6. The X-ray imaging apparatus as recited in claim 1, wherein
the SSD deriving means is SSD calculation means configured to calculate the SSD from a relative position between a model imitating the subject and the focal point of the X-ray tube.

7. The X-ray imaging apparatus as recited in claim 1,
wherein
the physical quantity is the SSD, and
the X-ray imaging apparatus further comprises:
a top board configured to place the subject thereon;
storage means configured to store a combination of a rotation operation amount of the X-ray tube about a body axis of the subject, a rotation operation amount of the X-ray tube in a body axis direction of the subject, and an operation amount of the X-ray tube in a direction perpendicular to a placement surface of the top board, which do not fall below a reference SSD in which imaging is allowed; and
display means configured to display the combination.

8. The X-ray imaging apparatus as recited in claim 1,
wherein
the control means notifies that the physical quantity at the time of imaging deviates from an imaging allowable range with reference to the reference physical quantity by using the comparison result by the comparing means.

9. The X-ray imaging apparatus as recited in claim 8, wherein
the physical quantity is the SSD,
the comparing means compares the SSD at the time of imaging and the reference SSD in which imaging is allowed, and
the control means makes a notification that the SSD at the time of imaging falls below the reference SSD when the SSD falls below the reference SSD and simultaneously perform a moving operation of the X-ray tube so that the SSD at the time of imaging does not fall below the reference SSD by using the comparison result by the comparing means.

10. The X-ray imaging apparatus as recited in claim 9, wherein
the control means makes the notification and simultaneously stops the rotation operation of the X-ray tube.

11. The X-ray imaging apparatus as recited in claim 9, wherein
the control means makes the notification and simultaneously retreats the X-ray tube so as to move the X-ray tube away from the subject.

12. The X-ray imaging apparatus as recited in claim 9, further comprising a top board for placing the subject thereon,
wherein the control means makes the notification and simultaneously translates the top in a direction perpendicular to a placement surface of the top board so that the subject is moved away from the X-ray tube to continuously perform a rotation operation of the X-ray tube in a same direction as a direction immediately before the notification.

13. The X-ray imaging apparatus as recited in claim 9, wherein
when “n” is an integer of 2 or more and “k” is an integer satisfying 2<k<n,
the setting means sets values of the reference SSD in descending order in a plurality of stages of A₁, A₂, . . . , Aₖ, . . . , A₍ₙ₋₁₎, Aₙ, and
the control means
performs a process of notifying that the SSD at the time of imaging falls below the reference SSD(A₁) when the SSD at the time of imaging falls below the reference the SSD(A₁) and simultaneously continuously performing the rotation operation of the X-ray tube until the SSD reaches the reference SSD(A₂) reset by the setting means,
thereafter repeatedly performs a process of notifying that the SSD at the time of imaging falls below the reference SSD(Aₖ) when the SSD at the same time of imaging falls below the reference SSD(A₍ₖ₋₁₎) and simultaneously continuously performing the rotation operation of the X-ray tube until the SSD reaches the reference SSD(A₍ₖ₋₁₎) reset by the setting means, and
notifies that the SSD at the time of imaging falls below the reference SSD(Aₙ) when the SSD falls below the reference SSD(Aₙ) and simultaneously stops the rotation operation of the X-ray tube.

14. The X-ray imaging apparatus as recited in claim 9, further comprising selection means configured to select one moving operation mode from a plurality of moving operation modes of the X-ray tube, the X-ray detector for detecting X-rays and the top board for placing the subject thereof,
wherein the control means performs the notification and simultaneously performs the moving operation of the X-ray tube, the X-ray detector, or the top board according to the moving operation mode selected by the selection means.

15. The X-ray imaging apparatus as recited in claim 9, further comprising selection means configured to select one moving operation mode from a plurality of moving operation modes of the X-ray tube, the X-ray detector for detecting X-rays, or the top board for placing the subject thereon,
wherein after performing the selection by the selection means after the notification, the control means performs the moving operation of the X-ray tube, the X-ray detector, and the top board according to the moving operation mode selected by the selection means.
